(19) United States
(12) Patent Application Publication
(10) Pub. No.: US 2025/0281166 A1
(43) Pub. Date: Sep. 11, 2025

(54) SYSTEMS FOR REDUCING FRICTION IN ENDOVASCULAR DEVICES

(71) Applicant: Rapid Medical Ltd., Yokneam (IL)

(72) Inventors: Eyal KAUFMAN, Yokneam (IL); Noam PERLIS, Yokneam (IL); Matan GEDULTER, Yokneam (IL)

(73) Assignee: Rapid Medical Ltd., Yokneam (IL)

(21) Appl. No.: 18/597,546

(22) Filed: Mar. 6, 2024

(51) Int. Cl. A61B 17/00

(52) U.S. Cl. CPC A61B 17/00234; A61B 2017/00309; A61B 2017/00323; A61B 2017/00367; A61B 2017/00831

(57) ABSTRACT

An endovascular device for navigation of a blood vessel is disclosed. The endovascular device includes a tube with a selectively bendable portion, a control element extending through at least a portion of the tube, wherein movement of the control element relative to the tube is configured to cause the selectively bendable portion of the tube to bend, and a spacer disposed within the tube and placed between the tube and the control element, the spacer comprising at least one wire formed in a coil and configured to reduce sliding friction between the tube and the control element, and wherein the coil has a pitch at least 1.2 times a diameter of the wire.

### BACKGROUND

This disclosure relates to the field of endovascular medical devices. Specifically, this disclosure is related to endovascular devices intended to pass through a blood vessel of a patient to a target area inside the patient's body to perform a medical procedure.

An example of an endovascular treatment of the type relevant to this disclosure is the use of an endovascular device such as a catheter to treat narrowing, blockage, or hemorrhage in a blood vessel, including neurovascular, cardiovascular, and peripheral vasculatures. For instance, treatment of an acute stroke caused by a blockage of a blood vessel in the brain typically comprises either the intra-arterial administration of thrombolytic drugs such as recombinant tissue plasminogen activator (rtPA), mechanical removal of the blockage, or a combination of the two. These interventional treatments must occur within hours of the onset of symptoms. Both intra-arterial (IA) thrombolytic therapy and interventional thrombectomy involve accessing the blocked cerebral artery via endovascular techniques and devices.

Devices employed in these endovascular procedures may include balloons, snares, coils, stents, temporary stents (including those referred to as “stent-retrievers”), suctioning (e.g., of a blood clot with or without adjunct disruption of the clot (e.g., by use of aspiration catheters)), or a combination of these, depending on the condition to be treated and its anatomical location. Guide catheters, guide sheaths or guidewires are typically used to introduce, guide and/or position interventional devices within the peripheral, cardiovascular and neurovasculature systems from an arterial access site, typically the femoral or radial arteries. These medical devices are often used in a nested fashion, namely, a guidewire inside a microcatheter inside an intermediate catheter are advanced as an assembly to the target site.

Notably, for an endovascular device to work effectively, the device needs to be positioned as close as possible to the source of disruption, e.g., to a blood clot or a site of narrowing blood vessels. This may be challenging in anatomical areas in which the blood vessels comprise tortuous anatomy, such as the brain. Existing guidewire devices often require the medical practitioner to manually alter a curvature of a tip of the device before the device is inserted into the blood vessel, require the medical practitioner to replace the device by another more suitable guidewire during the procedure, or to remove the guidewire reshape it and reintroduce into the patient. Furthermore, these devices often have difficulty overcoming distal tortuous anatomy due to the lack of support or stiffness, especially for the distal ends of catheters, and lack of the ability to navigate through these rough vessels.

### BRIEF SUMMARY OF THE INVENTION

In a first embodiment, an endovascular device includes a tube comprising a selectively bendable portion, wherein at least a portion of the tube is configured to be inserted into a blood vessel; a plug disposed at a distal end of the tube, wherein the plug has a rounded tip configured to be inserted into the blood vessel; a washer disposed between the plug and the tube, the washer fixed with respect to the tube; and a control element extending through at least a portion of the tube, a distal end of the control element connected to the washer, wherein movement of the control element relative to the tube is configured to cause the selectively bendable portion of the tube to bend.

In a second embodiment an endovascular device includes a tube comprising a selectively bendable portion; a plurality of slots formed in the tube, each of the slots disposed partially around the circumference of the tube; a control element extending through the tube, the control element connected to a distal portion of the selectively bendable portion, wherein movement of the control element with respect to the tube is configured to bend the selectively bendable portion; and a spacer disposed within the tube and placed between the tube and the control element, the spacer configured to reduce sliding friction between the tube and the control element.

A method for controlling stiffness of an endovascular device according to an embodiment of the present disclosure begins with receiving a medical image captured during an endovascular procedure. The medical image is analyzed to detect a target area where a path of a vascular system to a target vessel changes direction. The medical image is also evaluated to detect an endovascular device extending along the path. It is then determined if a particular portion of the endovascular device is positioned at the target area, and the curvature of the particular portion of the endovascular device is controlled as needed.

A method of navigating a catheter through a blood vessel according to an embodiment of the present disclosure includes steering an endovascular device configured as a guidewire through the blood vessel to a location of interest, wherein the endovascular device comprises a selectively bendable portion at a distal end of the endovascular device and positioning the endovascular device at the location of interest, wherein the location of interest comprises a position along a blood vessel. The method further includes orienting the distal end of the endovascular device by actuating the selectively bendable portion of the endovascular device to create a tip shape; and navigating the catheter through the blood vessel by advancing the catheter over the guidewire to the location of interest.

Certain aspects of the disclosure have other steps or elements in addition to or in place of those mentioned above. The steps or elements will become apparent to those skilled in the art from a reading of the following detailed description when taken with reference to the accompanying drawings.

### BRIEF DESCRIPTION OF THE DRAWINGS/FIGURES

The accompanying drawings, which are incorporated herein and form a part of the specification, illustrate the present disclosure and, together with the description, further serve to explain the principles thereof and to enable a person skilled in the pertinent art to make and use the same.

FIG. 1 is a perspective view of an endovascular device according to an embodiment.

FIG. 1A is a perspective view of an endovascular device according to an embodiment.

FIG. 2 is a perspective view of a distal portion of an endovascular device according to an embodiment.

FIG. 2A is a side view of a portion of an endovascular device according to an embodiment.

FIG. 2B is a side view of a portion of an endovascular device according to an embodiment.

FIG. 3 is a partial section view of the endovascular device of FIG. 2.

FIG. 3A is a top and side view of an actuator of an endovascular device according to an embodiment.

FIG. 3B is a partial section view of the endovascular device according to an embodiment.

FIG. 3C is a side view of an exemplary spacer for an endovascular device according to an embodiment.

FIG. 4 is a cross section of the endovascular device of FIG. 2.

FIG. 4A is a cross section schematic of an endovascular device according to an embodiment.

FIG. 4B is a cross section view of the endovascular device of FIG. 3B.

FIG. 4C is a top and side view of an actuator of an endovascular device according to an embodiment.

FIG. 5 is a perspective view of a proximal portion an endovascular device according to an embodiment.

FIG. 6 is cross section of the endovascular device of FIG. 5.

FIG. 7 is a partial section view of a proximal portion of an endovascular device according to an embodiment.

FIG. 7A is a detail view of a portion of an endovascular device according to an embodiment.

FIG. 8 is a perspective view of an endovascular device according to an embodiment.

FIG. 9 is a partial section view of the endovascular device of FIG. 8.

FIG. 10 is a cross section of a proximal portion of an endovascular device according to an embodiment.

FIG. 11 is a diagram of a flowchart showing a method of using an endovascular device according to an embodiment.

FIG. 12 is a diagram of a flowchart showing a method of using an endovascular device according to an embodiment.

In the drawings, like reference numbers generally indicate identical or similar elements. Additionally, generally, the left-most digit(s) of a reference number identifies the drawing in which the reference number first appears.

### DETAILED DESCRIPTION

Reference will now be made in detail to representative embodiments illustrated in the accompanying drawings. References to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such a feature, structure, or characteristic in connection with other embodiments whether or not explicitly described.

Endovascular devices used to perform intravascular medical treatments need to be able to navigate through a blood vessel to reach the treatment site. This can be difficult in situations where the blood vessels in question comprise multiple turns because the typical endovascular device is not able to actively bend to navigate a twist or turn. Thus, an embodiment of the present disclosure is an endovascular device having a tube with a selectively bendable portion, where at least a portion of the tube is configured to be inserted into a blood vessel, and a control element configured to control a curvature of the selectively bendable portion to enable navigation of the tube through the blood vessel.

The bendable nature of the endovascular device has several benefits, including an improved ability to navigate curved blood vessels and to selectively and controllably change the shape of a portion of the endovascular device from a proximal location, which can be beneficial during certain endovascular procedures. Additional benefits are discussed below.

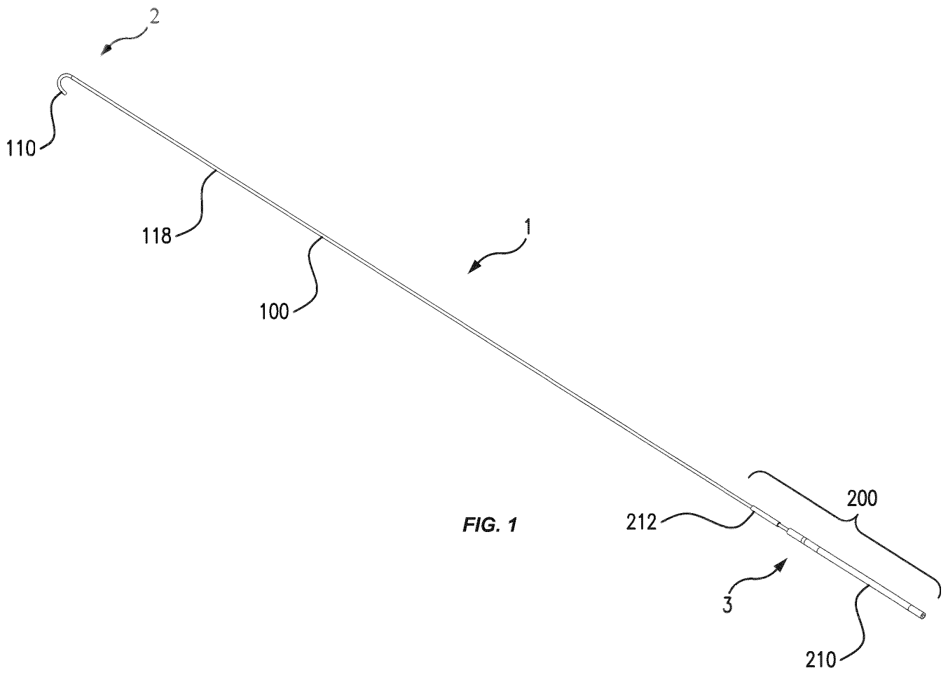

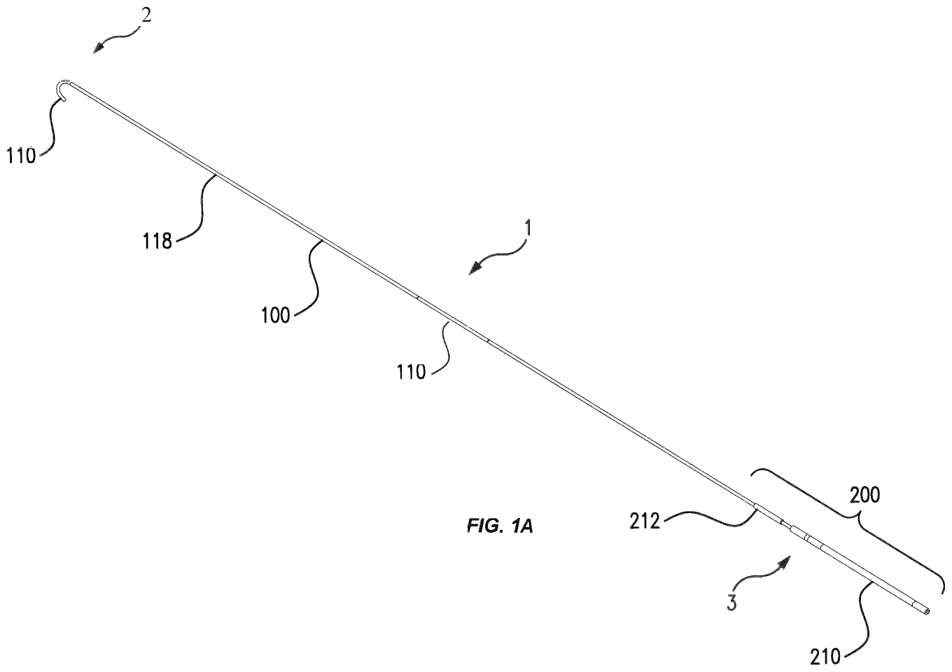

As seen in FIG. 1, an endovascular device 1 is formed as an approximately tubular device. Endovascular device 1 includes a distal end 2 and a proximal end 3 (also referred to as “distal tip” and “proximal tip”, respectively). Proximal end 3 is the end of tube 100 that generally remains outside of a patient and is manipulated by a user (e.g., a doctor or other medical professional). Distal end 2 is the end of tube 100 that is generally inserted into a blood vessel of the patient. In some embodiments, endovascular device 1 may have an overall length of between 1500 and 2500 millimeters (mm). In some embodiments, endovascular device 1 may have an overall length of about 2000 mm.

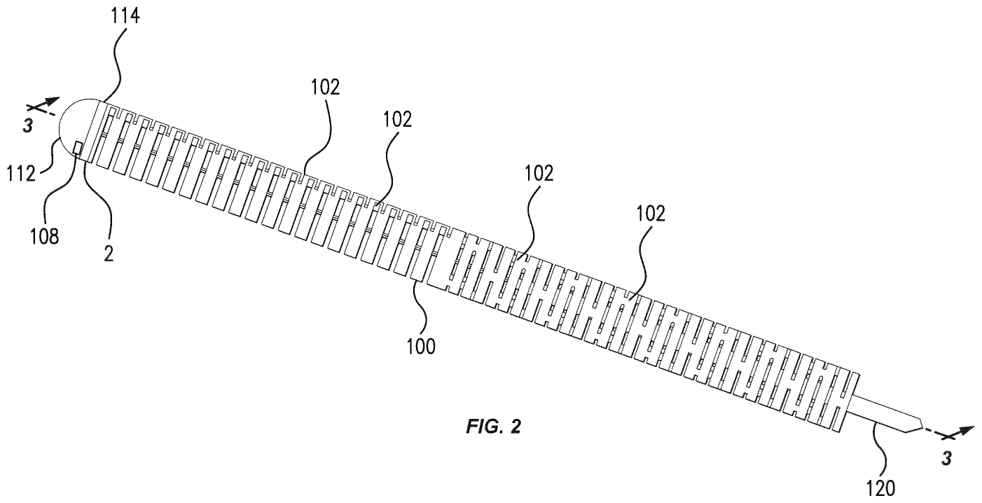

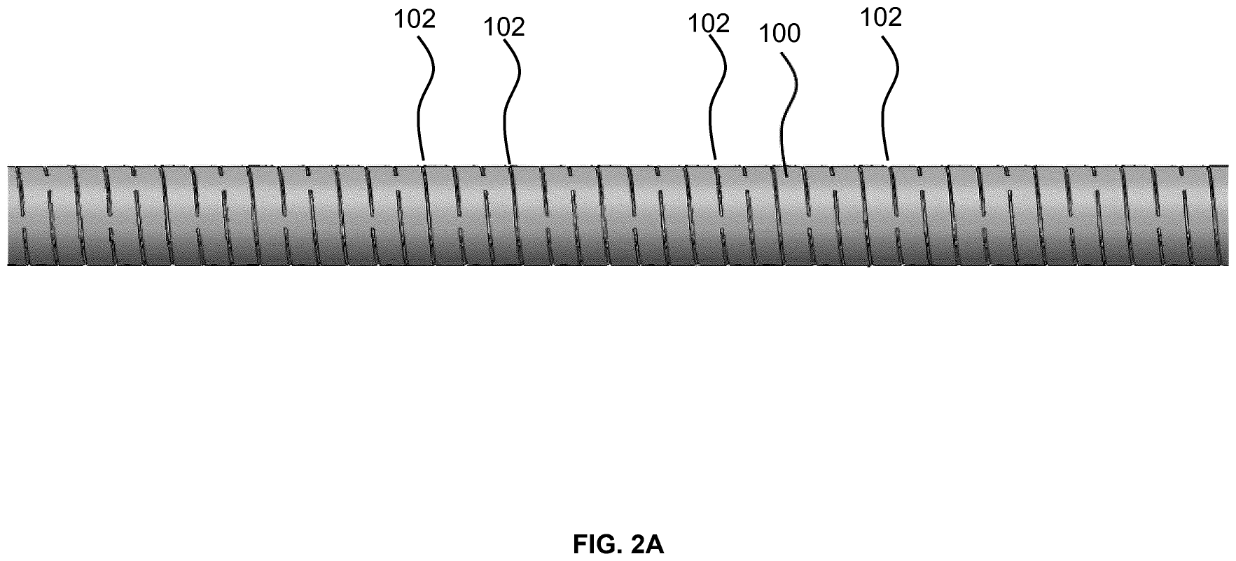

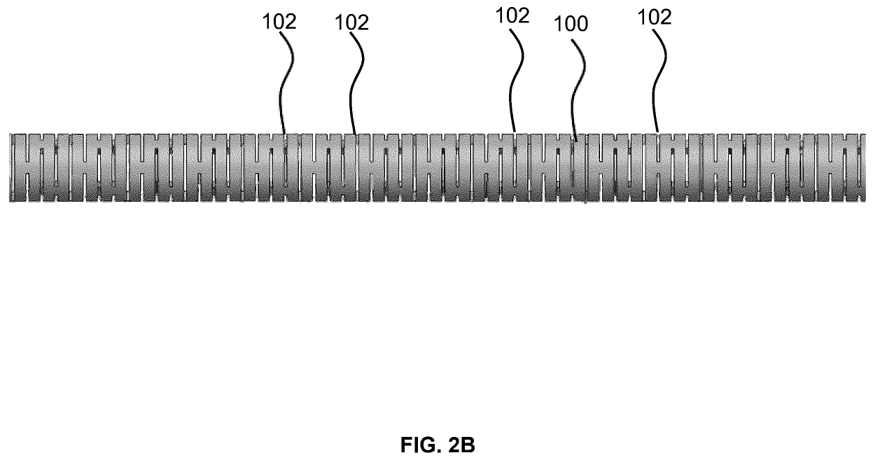

Endovascular device 1 is formed from a tube 100. Tube 100 makes for a body of endovascular device 1. As seen in FIGS. 1 and 2, in some embodiments tube 100 can be formed as a cylinder with a central opening. Tube 100 can be formed from any suitable material known in the art, such as metals or plastics, as discussed in detail below. As tube 100 is intended to be at least partially inserted into a blood vessel, the material of tube 100 should be biocompatible. Examples of materials for tube 100 (e.g., for the entire body of tube 100, or at least one of the distal end 2, the proximal end 3, or the intermediate tubular section including non-bending portion 118, therebetween, as well as any combinations thereof) are elastic and/or super-elastic polymers, super-elastic metals or various metals/alloys/oxides such, without being limited to, elastomers, silicon polymeric materials like Polydimethylsiloxane (PDMS), silicon adhesives, silicone rubbers, natural rubbers, thermoplastic elastomers, polyamide, polyimide, poly ethylene (PE), poly propylene (PP), polyether etherketone (PEEK), Acrylonitrile butadiene styrene (ABS), epoxys, polytetrafluoroethylene (PTFE), polyurethane, thermoplastic polyurethanes (TPU), Nylon, Polyether block amide (PeBax), Kevlar, stainless titanium, steel or stainless steel, nickel titanium alloy (Nitinol), nickel-chromium alloy, nickel-chromium-iron alloy, cobalt alloy, tungsten, cobalt, chrome, nickel, aluminum, copper, molybdenum or any combination thereof. According to a specific embodiment, tube 100 may be formed from stainless steel. According to some embodiments, tube 100 is configured as a monolithic, or single part, element. In alternative embodiments, tube 100 is configured to combine tubes of different materials (discussed in further detail hereinbelow). In some embodiments, tube 100 is configured to be elastically deformable along at least portions of its length, as will be explained below.

In some embodiments, tube 100 may have an outer diameter (OD) of between about 0.10 mm and about 1.10 mm. In some embodiments, tube 100 may have an OD of between about 0.30 mm and about 0.90 mm. In specific embodiments, tube 100 may have an OD of about 0.35 mm, about 0.45 mm, about 0.55 mm, about 0.65 mm, about 0.75 mm or about 0.80 mm. As mentioned above, tube 100 can be formed as a cylinder with a central opening. According to some embodiments, tube 100 may have an inner diameter (ID) of between about 0.10 mm and about 0.90 mm. In some embodiments, tube 100 may have an ID of between about 0.20 mm and about 0.70 mm. In specific embodiments, tube 100 may have an inner diameter of about 0.20 mm, about 0.30 mm, about 0.40 mm, about 0.50 mm or about 0.60 mm.

Endovascular device 1 includes at least one selectively bendable portion 110 (shown in the bent configuration in FIG. 1). As shown in FIG. 1, the bendable portion can be located at distal end 2, however, the bendable portion can be located at any other location along tube 100, depending on the needs and circumstances. The bendable portion is controlled by an actuator control 200 at proximal end 3, as discussed in detail herein below. As will be explained in detail below, embodiments such as those shown in FIG. 1A can have multiple selectively bendable portions 110.

Selectively bendable portion 110 is a portion of tube 100 that can be controllably bent with respect to the original axis of tube 100. As will be discussed further below, selectively bendable portions 110 can be controlled by a user to improve navigation of endovascular device through the blood vessel of a patient. In some embodiments, selectively bendable portion 110 can extend between about 5 mm and about 50 mm in the axial direction along tube 100. In some embodiments, selectively bendable portion 110 can extend between about 10 mm and about 30 mm in the axial direction along tube 100. In specific embodiments, selectively bendable portion 110 can extend about 12 mm, about 13 mm, about 14 mm, about 15 mm, about 16 mm, about 17 mm, about 18 mm, about 19 mm, about 20 mm or about 25 mm in the axial direction along tube 100.

Actuator control 200 is a control element that enables a user to achieve control of selectively bendable portion 110. Actuator control 200 includes a handle 210 and a grip 212 for controlling the curvature of selectively bendable portion 110.

Grip 212 is configured to allow a user grasp endovascular device 100. When the user moves the grip 212 linearly with respect to the patient, the endovascular device 100 can move deeper or shallower in a patient's blood vessel. When the user moves grip 212 torsionally, the tube 100 can rotate within a patient's blood vessel, effectively rotating an orientation of selectively bendable portion 110.

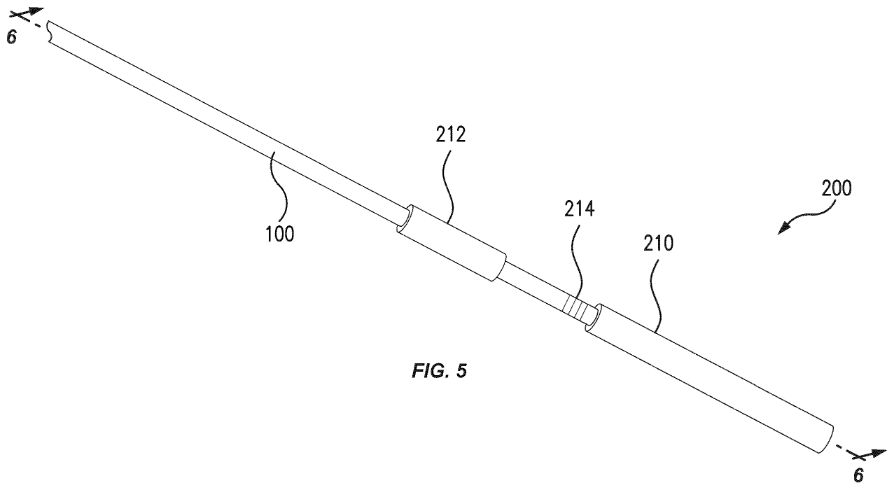

A user can cause a selectively bendable portion 110 to curve, for example, by changing a relative distance between handle 210 and grip 212. An interventional radiologist, for example, may grasp handle 210 with one hand and grip 212 with the other. By increasing the relative distance, selectively bendable portion 110 becomes more curved, e.g., a radius of curvature becomes shorter. And, by decreasing the relative distance, selectively bendable portion 110 becomes less curved, e.g., a radius of curvature becomes longer, until selectively bendable portion 110 is a straight extension of tube 100. In this way, a user, such as an interventional radiologist, is able to adjust selectively bendable portion 110 according to upcoming conditions in the patient's vascular pathway. How this curvature control may be achieved is described below with respect to FIGS. 5-8.

For example, when an artery or vein branches out in two directions, the user may move handle 210 with respect to grip 212 to create the desired curvature. Then the user may rotate grip 212 to orient the curvature of selectively bendable portion 110 towards the desired branch and may move grip 212 linearly to move distal end 2 of endovascular device 1 down the desired branch. In this way, the user can navigate distal end 2 of endovascular device 1 to a desired position within a patient's vascular system.

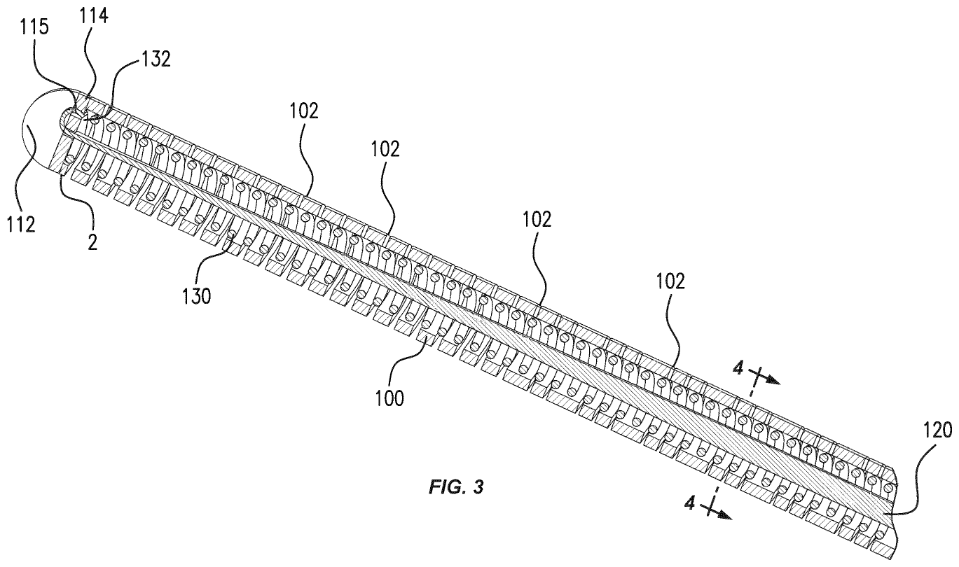

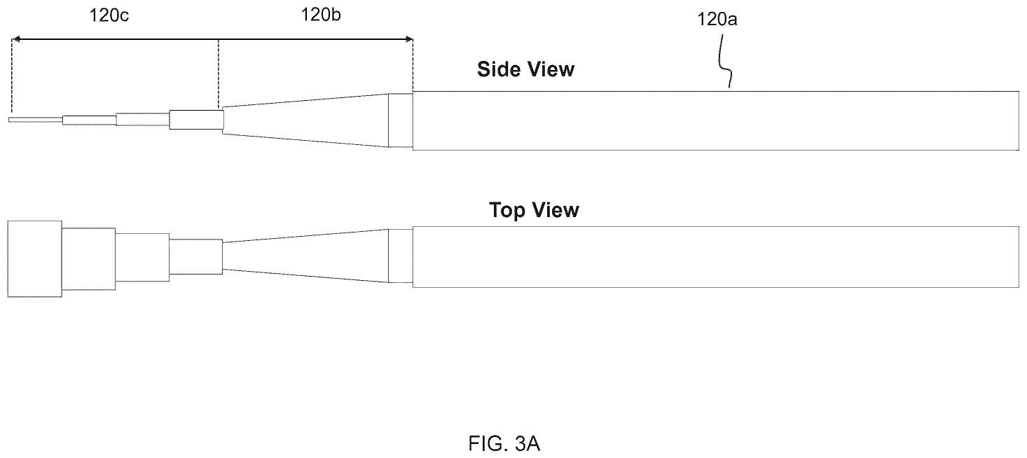

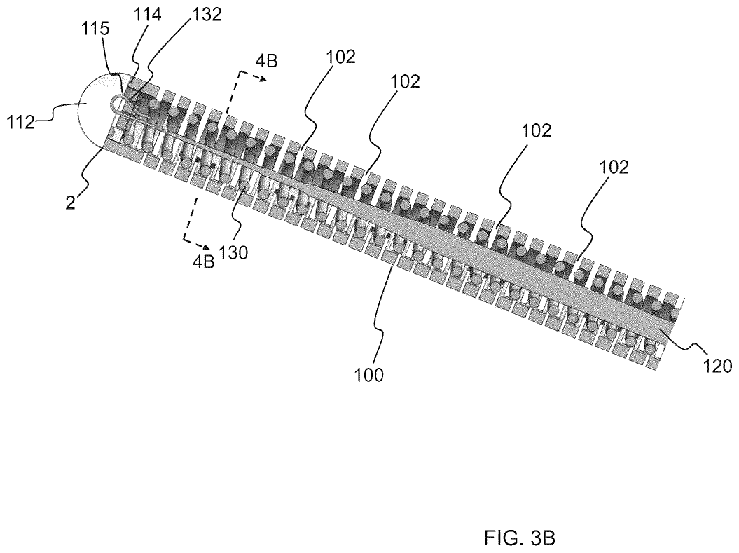

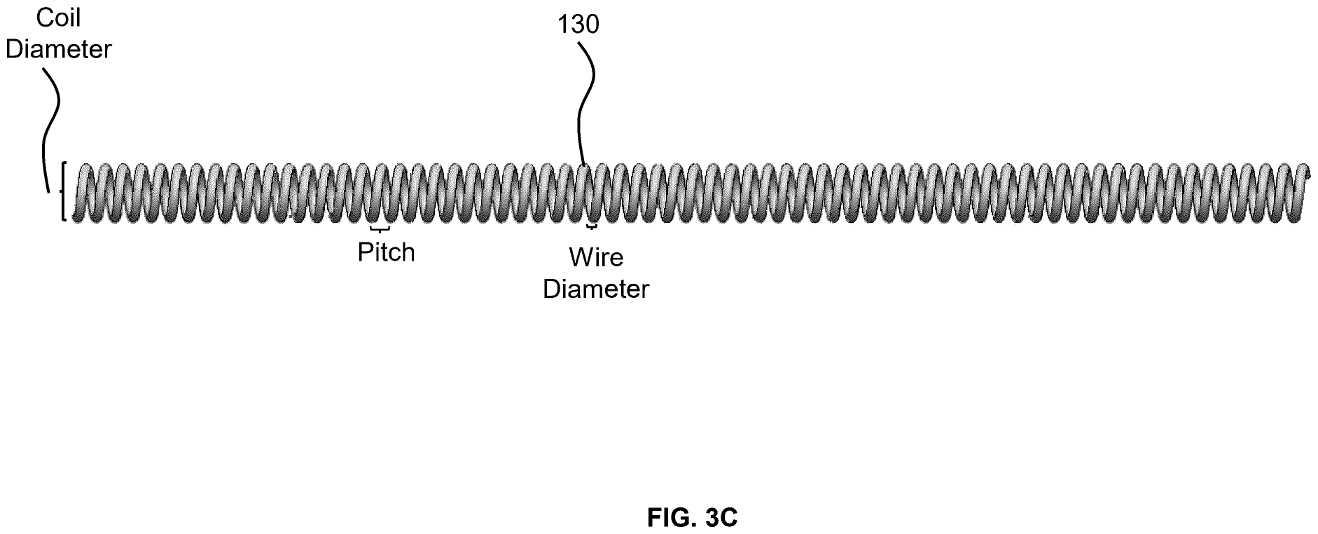

In some embodiments, as seen in FIGS. 2-3, openings 102 are formed in tube 100. As mentioned above, tube 100 may be made of any suitable material, including medical grade stainless steel. Openings 102 are portions removed from the material (e.g., cutouts). In some embodiments, these openings 102 are formed as slots in tube 100 that penetrate at least partially around a circumference of tube 100 from an exterior of tube 100 to an interior of tube 100. In some embodiments, openings 102 penetrate from the exterior of tube 100 to the interior of tube 100. In these embodiments, when endovascular device 1 is inserted into a patient's blood vessel, fluid may penetrate through openings 102. As shown in FIG. 2, openings 102 do not extend around the entire circumference of tube 100. Openings 102 can extend, for example, from between ten percent to ninety percent of the circumference of tube 100.

In some embodiments, openings 102 can be formed as a regular, rectangular shape projected onto the surface of tube 100 (as shown in FIG. 2). In these embodiments, for example, openings 102 can have a width in the axial direction of between about 0.01 mm and about 0.1 mm. According to specific embodiments, openings 102 can have a width in the axial direction of between about 0.02 mm and about 0.06 mm. Openings 102 can also be formed from different shapes projected onto the surface of tube 100, such as slits, circles, ovals, triangles, or any other desired polygonal shape. Openings 102 may be etched from tube 100, for example, using any manufacturing methods known in the art, such as by cutting or grinding, for example, with a disc, with a semiconductor dicing blade or using laser cutting.

By removing material from tube 100, openings 102 reduce the stiffness or resistance to bending of tube 100. Thus, openings 102 can be used to create more flexible or bendable portions of tube 100. These are portions of tube 100 that are more susceptible to being controllably bent, as will be discussed below. Changing the extent of openings 102 can therefore be used to control flexibility of tube 100. For example, increasing the extent, by increasing size, number, shape, or density of openings 102 will reduce the stiffness of tube 100, or increase the tendency to bend. Conversely, reducing the extent by reducing the size, number, shape, or density of openings 102 increases the stiffness of tube 100, or decreases the tendency to bend. Thus, as shown in FIG. 1, selectively bendable portion 110 of tube 100 has openings 102, while a second portion of tube 100 has no openings 102. Additionally, tube 100 may comprise a third portion (e.g., a non-bending portion 118 as further discussed below) comprising openings 102 but of reduced size, number, or density as compared to the selectively bendable portion 110. The third portion of tube 100 may be located anywhere along the tube, such as between the selectively bendable portion 110 and the second portion of tube 100, and provides a degree of softness to the tube at any required location depending on the needs and circumstances of the device.

It should be noted that tube 100 may be able to bend to some degree without any openings 102 present. But, the addition of openings 102 allows for the creation of targeted selectively bendable portions 110. In some embodiments, as shown in FIG. 2, openings 102 have identical dimensions, but have variable spacing between them. For example, openings 102 disposed in selectively bendable portion 110 may have a spacing of about 0.03 mm to about 0.2 mm (e.g., pitch of about 0.06 mm to about 0.3 mm). According to a specific embodiment, openings 102 disposed in selectively bendable portion 110 may have a spacing of about 0.08 mm to about 0.06 mm (e.g., pitch of about 0.12 mm to about 0.16 mm). Openings 102 in a separate, more proximal portion of tube 100 (such as in a third portion discussed above) are smaller and may be spaced further apart, and may have a spacing of between about 0.03 mm to about 3 mm (e.g., pitch of about 0.06 mm to about 3 mm).

Another example of changing the extent of openings 102 is changes in spacing between openings 102. Larger spacing between openings 102 would increase the stiffness of tube 100, while smaller spacing between openings 102 decreases the stiffness of tube 100. This technique can be used to create variable gradients of stiffness to tailor the stiffness of tube 100 as desired. For example, in some embodiments, the spacing or pitch of openings 102 in selectively bendable portion 110 is smaller at the end of selectively bendable portion 110 that is closest to distal end 2. In these embodiments, the spacing increases towards the end of selectively bendable portion 110 that is closer to proximal end 3. In this way, selectively bendable portion 110 is biased to be more flexible nearer distal end 2, which is the end that is inserted into the blood vessel first. The rate of change in spacing can be constant, or can be variable to tailor the stiffness change as needed. In some embodiments, there are no openings 102 for a predetermined distance from proximal end 3 of tube 100. This can be desirable to increase the stiffness of the proximal portion of tube 100. This portion of tube 100 may also be the portion of tube 100 intended to remain outside of the patient during an endovascular procedure.

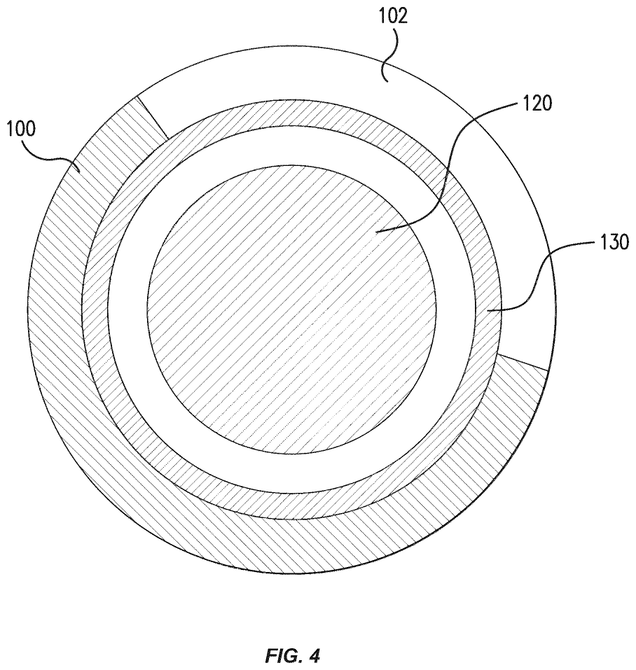

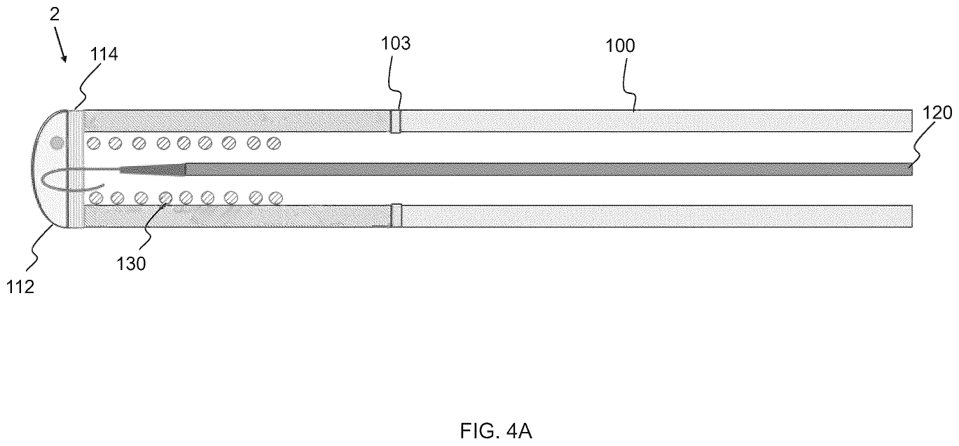

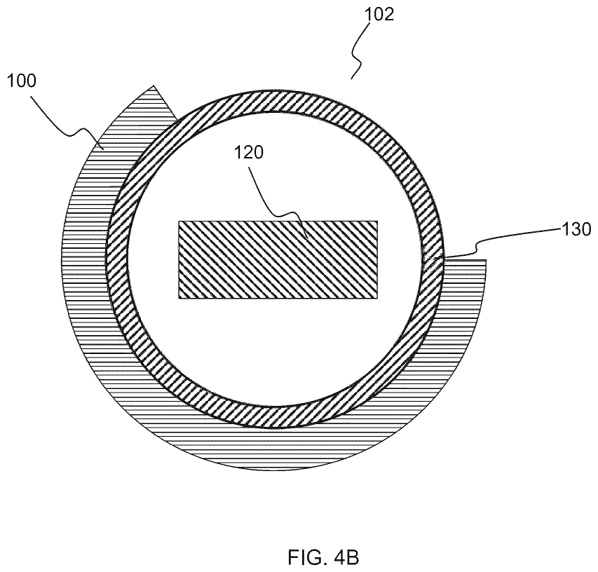

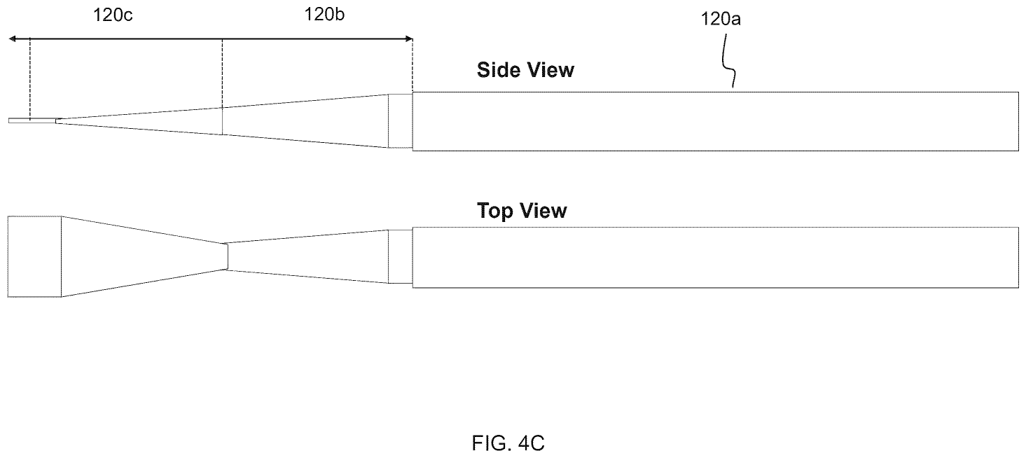

In some embodiments, the design or material of tube 100 can also be used to adjust flexibility of tube 100. For example, the cross-section dimensions or shape of tube 100 may be altered to increase or decrease flexibility. Changing the material of tube 100 can also change flexibility. For example, as shown in FIG. 4A, two or more portions of tube 100 may be made from different materials, which can be used to alter flexibility. A stiffer material will make the corresponding portion of tube 100 less flexible, for example. This can be useful when flexibility changes are desired without changing external dimensions of tube 100. These different materials can be joined together at a connection 103. Connection 103 can be, for example, any suitable connection technique including, but not limited to, adhesives, welding, or use of a mechanical connector such as a ring or sleeve (used alone or in combination with other connecting techniques).

As seen in FIGS. 1-3, in some embodiments there is a single selectively bendable portion 110 disposed at distal end 2 of tube 100. This enables a portion of tube 100 at distal end 2 to be more flexible, and thus selectively bendable. Selectively bendable portion 110 may also be disposed at various other points along tube 100 as needed to increase flexibility and control. Accordingly, selectively bendable portion 110 may be disposed at a distal portion of endovascular device 1 which is not the distal end 2, such as up to about 150 mm from the distal end 2. As seen in FIGS. 2-3, in some embodiments this distally located, selectively bendable portion 110 is terminated at distal end 2 with a plug 112. Plug 112 acts to finish or seal distal end 2. Plug 112 is generally an atraumatic and non-sharp tip which typically has a rounded, oval, or similar shape to improve the ability of tube 100 to transit inside a blood vessel in an atraumatic manner. A washer 114 is disposed between selectively bendable portion 110 and plug 112. Plug 112 and washer 114 can be fixedly connected to tube 100 by any suitable method, including but not limited to, welding, soldering, brazing, adhesive, or mechanical connections such as fasteners or crimping. Both plug 112 and washer 114 can be made from any suitable biocompatible material, including metals, metal alloys/oxides, adhesives, silicones, and plastics, or any combination thereof. Materials opaque to X-rays, such as platinum, gold, tungsten, tantalum or the like, may be incorporated into the plug or washer, to act as a fluoroscopic marker to aid in visualization while navigating the device in a blood vessel.

In some embodiments, selectively bendable portion 110 is the only portion of tube 100 that can be controllably actuated or bent. Thus, there can be one or more non-bending portions 118 that are disposed throughout tube 100. These non-bending portions 118 can be disposed immediately adjacent to selectively bendable portion 110, or can be spaced apart from selectively bendable portion 110. It should be noted that non-bending portions 118 may, and often will, still have some ability to passively flex and bend depending on the stiffness of tube 100 in non-bending portions 118. Techniques for tailoring this stiffness are discussed below.

As seen in FIGS. 3-4, for example, endovascular device 1 can include a control element 120 (also referred to as an actuator) disposed inside tube 100. Control element 120 can take the form of any suitably stiff element that can freely slide inside tube 100. Sufficient stiffness is needed to prevent control element 120 from buckling excessively when it is put in compression (during the straightening of selectively bendable portion 110). However, control element 120 must also be sufficiently flexible such that it does not render tube 100 too inflexible. The stiffness of control element 120 also affects the flexibility of selectively bendable portion 110, and thus the portion of control element 120 that transitions through selectively bendable portion 110 must be designed to allow for selectively bendable portion 110 to bend as desired. Control element 120 can be made of any suitable biocompatible material, including metals, metal alloys and plastics, or any combination thereof. According to one embodiment, control element 120 can be a solid wire, a multi-filament wire, a string, or a thread. For example, control element 120 can be a solid wire formed from any material known in the art, such as but not limited to, nitinol alloy, stainless steel, and a plastic material, or any combination thereof. For example, as shown in FIGS. 1-4, control element 120 can be formed as a solid wire that is sized to fit inside tube 100. Control element 120 can also take the form of a tube, as will be discussed below.

The function of control element 120 is to transmit force to selectively bend or straighten selectively bendable portion 110. This is accomplished by fixing the distal end of control element 120 to the distal end of the selectively bendable portion 110 (or to a different attachment point of the selectively bendable portion 110 depending on the needs and circumstances of the device). According to one embodiment, the distal end of control element 120 is fixed to washer 114, which is in turn fixed to an end of selectively bendable portion 110. Because selectively bendable portion 110 may be placed anywhere along tube 100, washer 114 may be located at different locations along tube 100 depending on where selectively bendable portion 110 begins and ends. As seen in FIGS. 3, 3B and 4A, in an embodiment, control element 120 passes through an opening in washer 114 and curves to fit into a groove 115 in washer 114. For example, as shown in FIGS. 3 and 3B, control element 120 extends through washer 114 traveling in an axial direction, and then curves approximately 180 degrees back to fit into groove 115, forming a u-shaped curve. In this way, control element 120 is fixed both axially and rotationally to washer 114 at distal end 2 of endovascular device 1. Examples of these types of curves are shown in both FIGS. 3, 3B and 4A. In addition to passing through washer 114 as described, control element 120 may also be secured by adhesives and mechanical techniques such as a friction fit or the formation of additional bends/angles in control element 120. This manner of connecting control element 120 to washer 114 also improves the connection between tube 100 and control element 120 because washer 114 substantially reduces the possibility of control element 120 separating from its fixed position with respect to tube 100.

Control element 120 can otherwise slide freely inside tube 100 to transmit force from actuator control 200 to washer 114 at distal end 2. From the straightened position shown in FIG. 2, retraction of control element 120 with respect to tube 100 towards proximal end 3 will result in selectively bendable portion 110 bending to accommodate the shortened length of control element 120 (as seen in FIG. 1). Conversely, extension of control element 120 towards distal end 2 will result in straightening of selectively bendable portion 110. More particularly, in an embodiment, movement of handle 210 in a proximate direction may increase tension on control element 120 causing curvature of selectively bendable portion 110, and movement of handle 210 in a distal direction may reduce tension on control element 120 causing straightening of selectively bendable portion 110.

In some embodiments, control element 120 can be configured to increase the tendency for selectively bendable portion 110 to bend in a desired direction or directions. This is accomplished by altering the shape of control element 120 to increase its tendency to bend in a given direction or directions. Thus, control element 120 can have any lateral cross-section (perpendicular to the longitudinal axis) such as round, elliptic, square, rectangular, and the like.

For example, in embodiments like the one of FIG. 4 control element 120 is formed from a solid wire with a varying cross section. Closer to proximal end 3, this embodiment of control element 120 has a circular cross section. As control element 120 continues in the distal direction, control element 120 flattens and has a rectangular cross section, as seen in FIG. 4. The rectangular cross section tends to bend in the direction perpendicular to the long sides of the rectangle, creating a preferred or biased bending direction for selectively bendable portion 110 that control element 120 is disposed within.

Additionally or alternatively, control element 120 can maintain the same cross section, but change in size to alter bending tendencies. For example, control element 120 may maintain a circular cross section, but may increase or decrease in diameter to increase or reduce stiffness, respectively. This type of control element 120 would not have a biased bending direction because the cross-sectional shape is maintained but would instead have lesser or greater bending tendencies depending on the dimensions of control element 120. The dimensions of these changed areas of control element 120 can be varied to achieve the desired resistance to bending when combined with the properties of tube 100. For example, an embodiment of this type of control element 120 can have different first, second, and third diameters at different locations along the length of control element 120.

For example, an embodiment of control element 120 is shown in FIG. 3A, which shows a side view and top view of control element 120. In this embodiment, control element 120 has a constant diameter portion 120a, a tapered portion 120b, and a flattened portion 120c. Constant diameter portion 120a has a cross-section of a predetermined diameter, which may correspond to parts of control element 120 that are closer to proximal end 3 of tube 100 when assembled into endovascular device 1. Tapered portion 120b has a diameter that constantly decreases as distance from constant diameter portion 120a increases. Flattened portion 120c has a rectangular cross-section (thus, it is flattened when compared to other parts of control element 120) that increases in width when viewed from the top as distance from tapered portion 120b increases. Flattened portion 120c can correspond to the selectively bendable portion 110, and thus can correspond to distal end 2 of tube 100. A skilled artisan will understand that flexibility of control element 120 will increase in all bending directions in tapered portion 120b as distance from constant diameter portion 120a increases because of the reduction in diameter. Bending in flattened portion 120c is biased towards bending in the direction in and out of the drawing with respect to the top view because of the flattened cross section shape. This embodiment of control element 120 is shown in tube 100 in FIG. 3B, and a cross-section of FIG. 3B is shown in FIG. 4B.

In some embodiments, constant diameter portion 120a ranges between about 0.1 and about 0.45, e.g., about 0.140 and about 0.180 mm in diameter. At its most proximal end, tapered portion 120b has the same diameter as constant diameter portion 120a. Tapered portion 120b can taper down to a diameter of between about 0.065 mm and about 0.2 mm. The taper may be evenly distributed (a linear taper) along the axial length of tapered portion 120b, or can be unevenly distributed. Flattened portion 120c may have a width dimension that begins at the smallest diameter of tapered portion 120b and increases up to the diameter of constant diameter portion 120a. As seen in FIG. 3A, there can be four steps of increasing width, with the first (most proximal) step ranging from about 0.100 mm to about 0.170 mm, the second step ranging from about 0.110 mm to about 0.180 mm, the third step ranging from about 0.120 mm to about 0.190 mm, and the fourth step ranging from about 0.130 mm to about 0.200 mm. Here, the width dimension is being defined as the dimension in the top to bottom direction of FIG. 3A with respect to the lower top view. It should be understood that there can be more or less steps in flattened portion 120c as needed or desired to adjust flexibility. Flattened portion 120c can also be flatted and expand in width in a gradual, non-stepwise fashion, as shown in FIG. 4C.

Biased bending as discussed above can also be achieved by distributing the open areas caused by openings 102 unevenly with respect to the circumference of tube 100. For example, making openings 102 larger on one side of tube 100 would bias tube 100 to bend in the direction of that side. Thus, having asymmetric slots having openings with a greater extent (e.g., more open area) facing in a first radial direction and slots with a smaller extent (e.g., less open area) facing in a second radial direction (e.g., direction opposite the first radial direction), biases the bending of the selectively bendable portion 110 in the first direction when the control element 120 is moved by actuator control 200. Using any combination of these methods to bias the bending of tube 100 has the advantages of providing predictable bending for the user of endovascular device 1. This is particularly helpful when distal end 2 of tube 100 has been inserted into a blood vessel and is not visible. The orientation of the remainder of tube 100 (present outside of the blood vessel) allows the user to understand the direction that the distal end of tube 100 with such a bias will bend with respect to the blood vessel.

In some embodiments, there can be two or more selectively bendable portions of tube 100. These selectively bendable portions of tube 100 may include biased bending region accomplished by having an asymmetrical distribution of openings 102 as discussed above.

The same tube 100 having asymmetrical distribution of openings 102 at the one or more selectively bendable portions of tube 100 can also have a symmetrical set of openings disposed in a different portion of tube 100. These symmetrical openings 100 improve flexibility of tube 100 without introducing any directional bias. Finally, the same tube 100 may have a portion without any openings 102, for example, on the portion of tube 100 intended to remain outside of the patient during the procedure.

For example, in some embodiments there may be selectively bendable portion 110 disposed at distal end 2 with asymmetrical openings as discussed above. Moving proximally from selectively bendable portion 110, there is a non-bending portion 118 that has openings 102 disposed symmetrically about the circumference of tube 100 to increase flexibility of tube 100. In this embodiment, openings 102 in non-bending portion 118 have an increasing spacing or pitch in the axial direction as non-bending portion 118 progresses axially towards proximal end 3. This has the effect of gradually increasing stiffness of tube 100 in the proximal direction. Finally, a portion of tube 100 that is proximal to non-bending portion 118 has no openings 102. These features provide the endovascular device with a soft, flexible and controllable distal section which is optimal for navigation in torturous anatomy, while providing support, shape retention and maintaining optimum torque transmission and tailored pushability in the more proximal sections.

In some embodiments, as shown in FIG. 2 and discussed above, the symmetrical openings 102 are formed as a slot (e.g., rectangular slot) that is projected or wrapped around some portion of the circumference of tube 100. In these embodiments, this portion may be anywhere between 0 and 350 degrees of the circumference of tube 100. The slot that is projected can be between 0.01 mm and 0.1 mm in width, and the pitch or spacing between slots (openings 102) can be between 0.04 mm and 50 mm. The orientation of the openings 102 can also be varied in different ways. As shown in FIG. 2, the ends of the openings 102 can be located at varying positions in terms of rotation about the axis of tube 100. This means that the solid portion of tube 100 between the ends of each opening 102 can be offset rotationally with respect to adjacent openings 102, as shown in FIGS. 2A and 2B. In some embodiments, this results in openings 102 being interleaved rotationally. In some embodiments, this offset can be arranged in a pattern such that each opening 102 is rotated a specific predetermined angular amount with respect to adjacent openings 102. According to one embodiment, each opening 102 may be rotated 0 to 350 degrees, for example, about 45 degrees, about 60 degrees, about 90 degrees, about 120 degrees, about 150 degrees, about 180 degrees, etc., with respect to the adjacent openings 102, which means that the openings 102 would form a repeating pattern in terms of their rotational alignment. Any desirable symmetrical or asymmetrical rotational alignment of openings 102 is possible. That is, the predetermined amount of rotational offset can be the same or can vary as desired between adjacent openings 102. In some embodiments, the plurality of slots comprises at least two slots (e.g., at least 3, 4, 5, 6, 7, 8, 9, 10, or more slots) dispersed along the tube, wherein each of the slots is rotationally interleaved with an adjacent slot.

In other embodiments, opening 102 can be inclined with respect to the axis of tube 100 at an angle between 0 and 90 degrees. This angle can be measured with slots that have at least one elongated or linear side, such as slots that are formed from projected rectangles. That is, the angle is taken as the angle between the elongated side and the axis of tube 100. As seen in FIG. 2A, openings 102 can be formed at a desired angle instead of being perpendicular to the axis of tube 100 (as seen in FIG. 2, for example). Any desired angle can be used to slant openings 102, and the same discussion above with respect to staggering the solid portions of tube 100 applies here. In these embodiments, this portion may be anywhere between 0 and 350 degrees of the circumference of tube 100. The slot that is projected can be between 0.01 mm and 0.1 mm in width, and the pitch or spacing between slots (openings 102) can be between 0.04 mm and 50 mm. In some embodiments, openings 102 can include both inclined and perpendicular orientations in different sections of tube 100. For example, selectively bendable portion 110 may comprise perpendicular openings, and the non-bending portion 118 may comprise non-perpendicular openings. Alternatively, the selectively bendable portion 110 may comprise non-perpendicular openings, and the non-bending portion 118 may comprise perpendicular openings. Furthermore, different parts of the non-bending portion may comprise alternating perpendicular and non-perpendicular sections. These determinations can be made by a person of ordinary skill in the art, based on the teachings provided herein.

Movement of control element 120 within tube 100 can cause friction. To reduce the friction generated between control element 120 and tube 100, endovascular device 100 may include a spacer 130.

As seen in FIGS. 3 and 4, spacer 130 can be disposed between control element 120 and the inside of tube 100. Spacer 130 acts as a spacer and guide that confines the movement of control element 120 inside tube 100. In these embodiments, and as discussed in the following paragraphs, spacer 130 is described as a solid material. However, spacer 130 may also include a liquid lubrication element, or may only be a liquid lubrication. For example, an oil can be added to the interior of tube 100 (e.g., near distal end 2). This oil can reduce friction between control element 120 and tube 100. Examples of suitable oils can include, without limitation, a silicone oil.

Spacer 130 is fixed with respect to the interior of tube 100. Suitable means for fixing spacer 130 to tube 100 include, but are not limited to, welding, soldering, brazing, using adhesives, and mechanical connections. In some embodiments, a connector 132 fixes spacer 130 to tube 100. In other embodiments, spacer 130 can be fixed to washer 114 using any method known in the art, such as with an adhesive, which in turn means spacer 130 is fixed to tube 100 because washer 114 is fixed to tube 100. According to an alternative embodiment, spacer 130 is fixed to tube 100 in a location closer to distal end 2. Accordingly, spacer 130 may be fixed at any location along tube 100 so as to enable longitudinal movement of control element 120. According to one embodiment, spacer 130 can completely surround control element 120 circumferentially. Alternatively, spacer 130 can only partially surround control element 120 circumferentially. In some embodiments, spacer 130 extends the entire length of tube 100. In other embodiments, spacer 130 extends only a portion of the axial length of tube 100. In other embodiments, spacer 130 extends the portion of the selectively bendable portion of tube 100. In some embodiments, spacer 130 extends 1 to 50 percent of the axial length of tube 100. In other embodiments, spacer 130 extends 5 to 30 percent of the axial length of tube 100. In other embodiments, spacer 130 extends 10 to 30 percent of the axial length of tube 100. Furthermore, spacer 130 may be placed at any section along the tube, e.g. at the distal end of the tube, proximal end of the tube, or anywhere in between.

In some embodiments, spacer 130 can be formed as a continuous tube of material. In other embodiments, spacer 130 can be formed from a strip or wire that is wrapped in a spiral or coil inside tube 100. In other embodiments, spacer 130 can be formed from several strips of material or wires that are wrapped in a spiral or coil inside tube 100. Other possible forms of spacer 130 include separate strips of material running the length of tube 100, or discrete rings of material separated from each other. Spacer 130 may be formed from a biocompatible material that allows control element 120 to slide with respect to spacer 130. For example, but not limited to, spacer 130 can be made from a metal, a metal alloy/oxide, a silicone, and a plastic material, or any combination thereof. Exemplary materials of spacer 130 include nitinol, platinum, iridium, polytetrafluoroethylene (PTFE), a fluoropolymer, such as polytetrafluoroethylene.

In some embodiments, as shown for example in FIG. 3C, a coil-type spacer 130 is formed from at least one wire shaped into a coil. According to one embodiment, a coil-type spacer 130 is formed from a single wire. According to one embodiment, a coil-type spacer 130 is formed from two or more wires, e.g., from 2, 3, 4 or 5 wires. The wire or wires forming the coil typically have a diameter of between 0.020 mm to 0.200 mm. The coil of spacer 130 typically has a diameter (also referred to as “coil diameter”) of between 0.20 mm to 1.20 mm. The coil of spacer 130 has a pitch, which is the linear distance it takes the coil to complete a single rotation about its central axis and which can be measured by finding the linear distance between the same or common angular point on adjacent coils. Thus, the pitch is usually measured as the center wire to center wire. According to a one embodiment, this pitch can range from 1.2 to 20 times the wire diameter, e.g., 1.5 to 10 times the wire diameter, e.g., 1.5 to 5 times the wire diameter. According to a specific embodiment, coil 130 has a pitch at least 1.2 times a diameter of the wire. According to a specific embodiment, coil 130 has a pitch at least 1.5 times a diameter of the wire. According to a specific embodiment, coil 130 has a pitch at least 2 times a diameter of the wire. According to a specific embodiment, coil 130 has a pitch at least 4 times a diameter of the wire. These measurements, and particularly the pitch, have the benefit of ensuring that spacer 130 allows for bending of tube 100, particularly in selectively bendable portions 110. It should also be understood that coil-type spacers 130 may maintain the same dimensions (e.g., wire diameter, coil diameter, and pitch) throughout, or may vary these dimensions to achieve different effects, such as increased or decreased resistance to bending.

In some embodiments, the material selected for spacer 130 is radiopaque, which means spacer 130 is visible on an x-ray scan, or similar types of medical imaging, when placed in the body. This can be achieved by material selection, or by the addition of an additive or coating to spacer 130. For example, materials such as gold, platinum, tungsten, tantalum or the like, may be incorporated into spacer 130, to act as a fluoroscopic marker to aid in visualization. According to one embodiment, spacer 130 is at least partially formed from a material selected from the group consisting of a metal alloy and a fluoropolymer material to ensure spacer 130 is sufficiently radiopaque. In other embodiments, tube 100 can be radiopaque, either by material selection (as discussed above) or by the addition of an additive or coating (discussed below).

As seen in FIGS. 5-7, an actuator control 200 is disposed at proximal end 3 of tube 100. As described above, actuator control 200 allows a user to move control element 120 with respect to tube 100. This portion of tube 100 and actuator control 200 is generally intended to remain outside of the blood vessel and body of a patient.

In embodiments, actuator control 200 is formed from a tubular handle 210 that is disposed around proximal end 3 of tube 100. Handle 210 is configured to slide about the outside of tube 100. This enables handle 210 to move with respect to grip 212 as described above. Handle 210 extends distally along tube 100 to the distal end of tube 100. According to an embodiment, handle 210 may extend beyond proximal end 3 of tube 100.

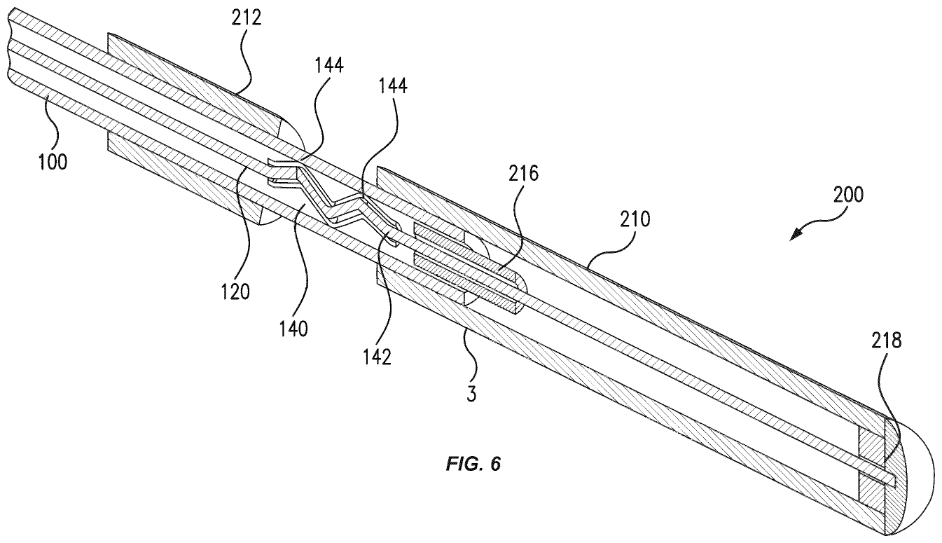

Control element 120 exits proximal end 3 of tube 100 into the center of handle 210, and is fixedly connected to the interior of handle 210 by any suitable means, including but not limited to, welding, soldering, brazing, adhesive, or mechanical connections such as fasteners or crimping. Thus, in some embodiments, control element 120 runs through the entire length of the tube and is connected to proximal end 3 of tube 100. For example, as seen in FIG. 6, control element 120 can extend to, and be fixed to, a plug 218 that closes the end of handle 210. In this way, movement of handle 210 with respect to tube 100 moves control element 120, which in turn bends selectively bendable portion 110.

As mentioned above, grip 212 is disposed around the exterior of tube 100 at a location distal to handle 210. Grip 212 is fixed to the exterior of tube 100 and is configured to act as a resting place for a hand of a user to better control the movement of handle 210. Both grip 212 and handle 210 can be formed to have a comfortable, non-slip gripping surface suitable for gripping by hand. Handle 210 may be formed from a composite construction with the portion of handle 210 adjacent to tube 100 formed from a material suitable for sliding against tube 100, such as a hard plastic, while the exterior-facing portion of handle 210 may be formed from a rubber or foam material for improved grip by a user.

A stopper 216 can be included as part of actuator control 200. Stopper 216 functions to stop handle 210 from being advanced too far distally, which is along tube 100. As seen in FIG. 6, in some embodiments, stopper 216 is a protrusion that is fixed to tube 100. This protrusion extends inside handle 210 a predetermined distance. Suitable means for fixing stopper 216 to tube 100 include, but are not limited to, welding, soldering, brazing, using adhesives, and mechanical connections. Friction element 140 contacts stopper 216, preventing handle 210 from advancing too far because friction element 140 is fixed to control element 120, which is in turn fixed to handle 210 (by way of plug 218). Other embodiments of stopper 216 can include structures such as protrusions or rings fixed to the interior of handle 210 that are configured to perform the same function.

As seen in FIG. 5, one or more markings 214 can be placed on an exterior of tube 100 such that the user can determine the extent of bending via the alignment of handle 210 and markings 214. Markings 214 can include a neutral marking that corresponds to a straightened position of selectively bendable portion 110.

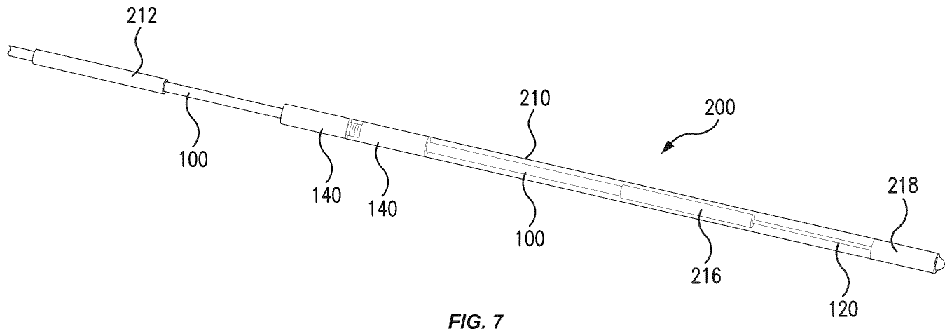

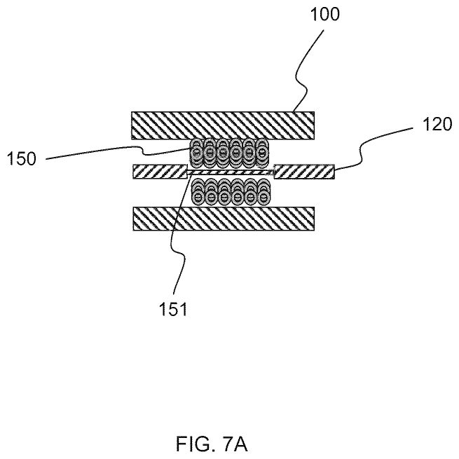

A key structure 150 can be formed in tube 100 to ensure that rotational torqueing of control element 120 is transmitted to tube 100. For example, a user may twist handle 210 with respect to the axis of tube 100, which in turn puts a rotational torque on control element 120. Key structure 150 ensures that this torque is transmitted to tube 100, which has the effect of rotating tube 100, depending on the torque applied by the user. For example, FIG. 7A shows an embodiment of key structure 150 that comprises material placed inside, and fixed to, tube 100. The opening in key structure 150 may be shaped to correspond to the shape of a narrow portion 151 of control element 120. The shape of narrow portion 151 (and corresponding key structure 150) is selected to prevent rotation of control element 120 with respect to tube 100. For example, control element 120 may be flattened into a rectangular cross-section at narrow portion 151. It will be appreciated that any suitable shape may be used to prevent rotation of control element 120. The material of key structure 150 can be any suitable material known in the art, including but not limited to, metals, plastics (such as polyether etherketone (PEEK)) and composite materials. Key structure 150 can be fixed to tube 100 using any suitable process, including but not limited to, adhesives, welding, soldering, and brazing.

As seen in FIG. 6, a friction element 140 can be disposed inside tube 100. Friction element 140 functions to increase the sliding friction affecting control element 120. This has at least two benefits. First, it allows the feel of moving control element 120 to be tailored to a desired value. This is important because a friction level that is too low will result in unintended movement of control element 120. A friction level that is too high can make control element 120 difficult to use with precision. In some embodiments, the desired friction level is approximately 0.5 to 2.0 Newtons, measured as the force needed to overcome sliding friction and move handle 210. The second advantage of tailoring sliding friction is that it enables control element 120 to be designed to remain in a given position once set. This is important because unwanted movement of control element 120 caused, for example, by movement of endovascular device 1 through a blood vessel can lead to issues such as movement of endovascular device 1 from a given position, such as when a catheter (e.g., an aspiration catheter) is being moved over a guidewire to reach a location of interest.

As seen in FIG. 6, in some embodiments, friction element 140 is formed from a tubular sleeve 142 that is placed around control element 120 inside tube 100. Tubular sleeve 142 is fixed to control element 120, for example by an adhesive, welding, a friction or press fit. Tubular sleeve 142 is sufficiently rigid to be shaped into different shapes. According to one embodiment, as seen in FIG. 6, tubular sleeve 142 may form several turns such that there are several distinct contact points 144 between tubular sleeve 142 and the inside of spacer 130 (or, in some embodiments, the inside of tube 100). These contact points 144 produce friction when control element 120 is moved.

The friction produced can be tailored by altering the total number of contact points 144, the surface area of contact at each contact point 144, and the pressure exerted between contact point 144 and the inside of spacer 130. For example, adding additional bends to tubular sleeve 142 results in more contact points 144, thereby increasing friction. Increasing the angle of each bend increases the surface area of contact point 144, also increasing friction.

Friction element 140 can be placed anywhere in tube 100, however it can be desirable to place friction element 140 in the portion of tube 100 that is intended to remain outside of the body (near proximal end 3). This ensures that the rigidity of friction element 140 does not affect how tube 100 bends inside the body.

As seen in FIG. 7, other embodiments of friction element 140 can adjust the friction between handle 210 and the outside of tube 100. In FIG. 7, a pair of tubes fixed to the inside of handle 210 from friction element 140. These tubes slide along the outside of tube 100 to set friction to the desired level. Other embodiments of friction element 140 can include structures such as discrete rings or washers that are fixed to control element 120 and contact the inner surface of spacer 130. Adjustment of friction forces can be accomplished in the same manner as discussed above by increasing the number or size of the contact points between the rings/washers and spacer 130. Other embodiments of friction element 140 can include structures such as rings, sleeves, or tubes fixed to the interior of spacer 130 that control element 120 passes through. In some embodiments, these structures can be integrated into spacer 130, as, for example, a narrow section of spacer 130 that applies friction to control element 120.

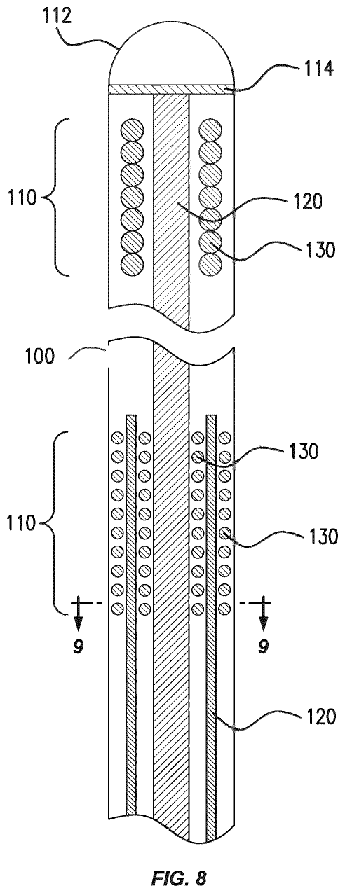

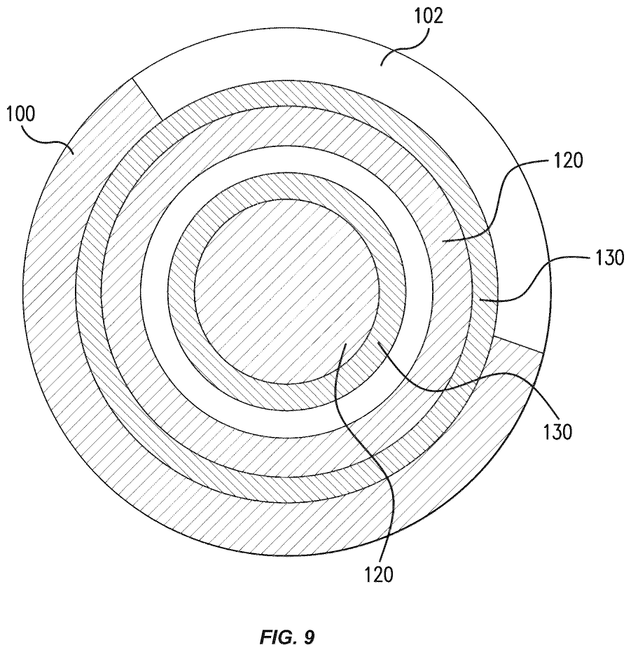

As seen in FIGS. 1A and 8-10, in some embodiments tube 100 comprises multiple selectively bendable portions 110. For example, there can be one, two, three, four, or more selectively bendable portions 110. The addition of more selectively bendable portions 110 can improve the ability for endovascular device 1 to navigate blood vessels because it adds more controlled directionality to tube 100. In some embodiments, as shown in FIG. 8, one selectively bendable portion 110 is disposed at distal end 2, as discussed above. A second selectively bendable portion 110 is disposed closer to proximal end 3 of tube 100. In some embodiments, the two selectively bendable portions 110 are adjacent to each other. In other embodiments, the two selectively bendable portions 110 are separated by a non-bending portion 118 of tube 100. In some embodiments, the separation is between about 5 mm and about 100 mm in an axial direction. According to some embodiments, the separation is between about 10 mm to about 50 mm in an axial direction. According to specific embodiments, the separation is of about 5 mm, about 10 mm, about 15 mm, about 20 mm, about 25 mm or about 30 mm in an axial direction. The discussion above with respect to biasing the bending of tube 100 applies equally multiple selectively bendable portions 110. For example, each selectively bendable portion 110 can be biased in a certain radial direction, which may or may not be the same direction as any other selectively bendable portions 110, using the techniques discussed above.

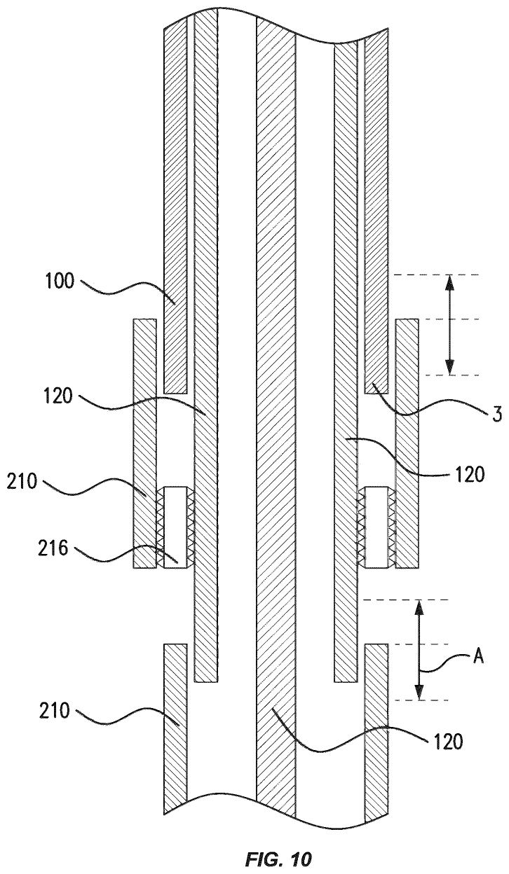

Each selectively bendable portion 110 includes its own control element 120 travelling through tube 100 and fixed to the distal end of the selectively bendable portion 110. Furthermore, each of the selectively bendable portions 110 may have a spacer 130, friction element 140, and actuator control 200 disposed at the proximal end 3, as seen in FIG. 10. This enables fully independent control of each selectively bendable portion 110. In some embodiments, there is a single grip 212 shared between the separate actuator controls 200. In other embodiments, there are two grips 212 each working in conjunction with one of the two actuator controls 200.

In the exemplary embodiment shown in FIGS. 1A and 8-10, control element 120 corresponding to the distal selectively bendable portion 110 is a wire (e.g., solid wire with a varying cross section as discussed in detail hereinabove). The wire control element 120 is fixed to the more distal selectively bendable portion 110 using the techniques discussed above. Furthermore, a spacer 130 may be placed between the wire control element 120 and the tube 100, such a spacer may also be fixed to the tube 100 using the techniques discussed above. Control element 120 corresponding to the more proximal selectively bendable portion 110 is a tube (also referred to as a “second tube”) that is disposed between the wire control element 120 and tube 100. A proximal spacer 130 may also be used between the tube control element 120 and tube 100. This configuration results in a symmetrical distribution of actuators 120, which reduces the impact to bending of tube 100. The tube control element 120 is fixed to the more proximal selectively bendable portion 110 using the techniques discussed above. Furthermore, proximal spacer 130 is fixed to the more proximal selectively bendable portion 110 using the techniques discussed above. However, there is not necessarily a washer 114 associated with the more proximal selectively bendable portion 110.

In embodiments where each control element 120 is formed as a wire, each control element 120 is routed through the center of tube 100, and then fixed to its respective selectively bendable portion 110 using the techniques discussed above. As discussed above, in some embodiments, it is desirable to fix control element 120 to the distal-most end of selectively bendable portion 110, but different attachment points are possible. In these embodiments, each control element 120 can have its own spacer 130, or a single spacer 130 can be used as discussed above.

Actuator controls 200 for devices comprising two or more selectively bendable portions 110 function in the same manner as the single selectively bendable portion 110 discussed above. However, as seen in FIG. 10, handles 210 for each actuator control 200 are staggered along tube 100 such that they can be independent actuated. For example, handle 210 associated with the proximal selectively bendable portion 110 can be placed more distally along tube 100. A connection between this handle 210 and its control element 120 can be accomplished via a slot or opening in tube 100. Handle 210 associated with the proximal selectively bendable portion 110 can be arranged as discussed above. Both handles 210 can have markings 214 configured as discussed above, and also can each have stoppers 216 that act as travel stops as discussed above.

In some embodiments, at least some of tube 100 may be coated with various substances to improve system performance. For example, an exterior surface of tube 100 intended for insertion into a patient may be entirely or partially equipped with an elastic or otherwise compliant, biocompatible coating or sheath to provide a smooth outer surface hydrophobic or hydrophilic, depending on the needs and circumstances. A coating material is selected to minimize sliding friction of the device during insertion and removal into a subject's body, and is substantially chemically inert in the in vivo vascular environment. According to one embodiment, the exterior surface of tube 100 may have a hydrophilic coating to reduce friction between tube 100 and a blood vessel. Examples of suitable coatings include, but are not limited to, polytetrafluoroethylene (PTFE), tetrafluoroethylene (TFE), urethane, polyurethane, thermoplastic polyurethanes (TPU), silicone Polyether block amide (PeBax), Nylon or polyethylene (PE), other polymers, polyurethane polymers, and elastomers are also suitable for coating. Additionally or alternatively, the coating material may be selected for its hydrophilic properties thus improving gliding in blood and navigability. Typically, this kind of coating is applied at the distal end 2 of tube 100 and extends up to 50 cm from the tip. Suitable coatings can be formed by any method known in the art, such as by dipping, spraying or wrapping and heat curing operations.

Other coating options include medications, which can be delivered into the body of the patient during operation of endovascular device 1. Such drugs can be used for treatment of a patient, to assist the efficacy of the procedure, or to avoid complications capable of evolving as a result of the procedure. For example, coatings of this type can include anti-restenotic drugs (including drugs which inhibit coagulation, such as anticoagulants, antithrombotic agents and antiplatelet agents like clopidogrel and heparin), anti-inflammatory agents, immune suppressants, antibiotics, antihyperlipidemic agents, antiproliferative agents and pro-endothelializing agents. An example of a detailed list of these types of coatings can be found in Tables 8 and 12 of Rykowska I. et al., Molecules (2020) 25:4624; doi:10.3390/molecules25204624, which is incorporated herein by reference.

In some embodiments, control element 120 can be coated to reduce friction. Suitable coatings for control element 120 include polymers and elastomers. For example, polytetrafluoroethylene (PTFE), tetrafluoroethylene (TFE), nylon, may be used to coat control element 120 to reduce friction when control element 120 moves with respect to tube 100. Suitable coatings can be formed by any method known in the art, as discussed above.

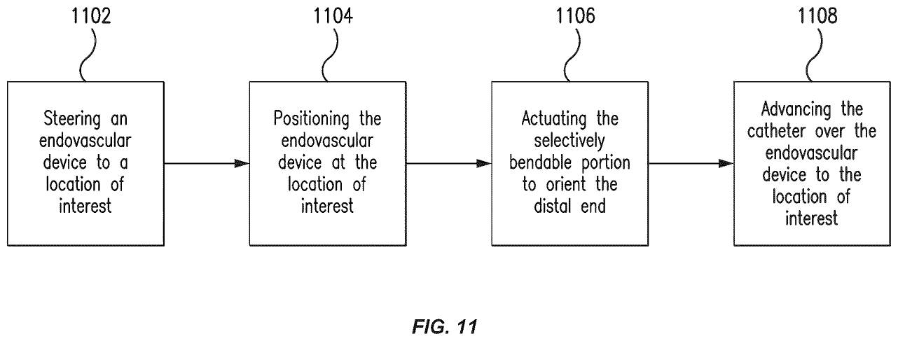

As diagramed in FIG. 11, a method of navigating a catheter begins with a step 1102 when distal end 2 of endovascular device 1 is inserted into a blood vessel by a user, such as a doctor or other medical professional performing a medical procedure. In some embodiments this blood vessel may be spatially separated from a location of interest. For example, the blood vessel may be located in the thigh of a patient or the wrist of a patient, and the location of interest may be located in the head, neck, stomach, chest or peripheral organs of the patient. The location of interest may be any location inside the body requiring a medical treatment to be performed with the assistance of endovascular device 1. For example, the location of interest may be a location comprising a blood clot, a stenosis, a contracted blood vessel (e.g., a vasospasm) or an aneurysm.

Next, in a step 1104, endovascular device 1 is advanced or steered through the blood vessel (or blood vessels) until it is positioned at the location of interest. This is accomplished, for example, by the user manipulating actuator control 200 to advance endovascular device 1. Selectively bendable portion 110 is controlled via actuator control 200 (which remains outside the patient's body) as needed to guide endovascular device 1 through the various curves present in the blood vessel.

Once distal end 2 has arrived at the location of interest, in a step 1106 selectively bendable portion 110 located at distal end 2 can be actuated to control the curvature of the tip to create a tip shape that orients the tip as needed. Additionally, or alternatively, the selectively bendable portion 110 located at distal end 2 can be actuated to increase support and/or to provide shape retention to orient the distal end of the endovascular device. For example, the tip shape can be oriented such that it is aligned or aimed at the location of interest. In some embodiments this tip shape can be a bent, or non-straightened shape, a J-shape, or a cobra shape. This orienting of the tip shape does not require any additional support to maintain its orientation. For example, no support is needed from the blood vessel, additional guidewires, additional catheters, or additional endovascular devices.

In some embodiments, endovascular device 1 is configured to function as a guidewire that can be used to guide a catheter through the blood vessel to the location of interest. Thus, in a step 1108, the catheter can be advanced through the blood vessels to perform the relevant medical procedure at the location of interest. For example, the catheter can be an aspiration catheter, and the location of interests can be at or adjacent to a blood clot. Upon arriving at the location of interest, the aspiration catheter can be used to remove the blood clot via suction or other suitable treatment. These embodiments of endovascular device 1 can also be used to guide other medical devices, such as additional guide wires, different types of catheters, or any other suitable systems to locations of interest accessible via a blood vessel in the body. For example, the additional device can be a stent retriever, which can be used to retrieve a blood clot from the blood vessel by applying the stent retriever through the microcatheter. According to another example, the catheter can be a microcatheter, and the location of interest may be a location of a clot, of an aneurysm, or of a narrowed blood vessel, such as a vasospasm or stenosis.

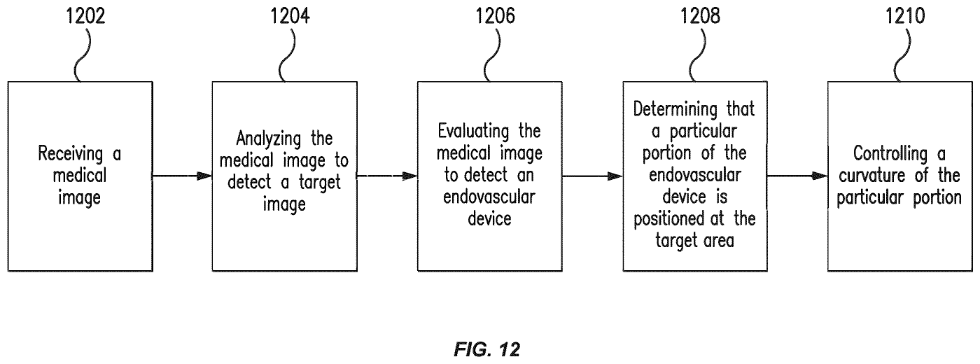

As diagramed in FIG. 12, a further method for navigating embodiments of endovascular device 1 through a vascular system begins with a step 1202, with receiving a medical image captured during an endovascular procedure. This medical image can be taken via any suitable medical imaging technique, such as but not limited to, X-ray, CT or MRI. The image can be stored on a suitable computer or other device.

In a step 1204, the medical image is analyzed to detect a target area where a path of the vascular system to a target blood vessel changes direction. For example, this may include a curve or twist in a blood vessel that requires additional steps for endovascular device 1 to navigate successfully. This can be accomplished, for example, by visual examination of the image, or via an appropriate algorithm run by a computer. For example, this may be accomplished with a computer vision model trained to recognize the blood vessel.

In a step 1206, the medical image is further analyzed to detect endovascular device 1 extending through the target blood vessel. This can be accomplished, for example, by visual examination of the image, or via an appropriate algorithm run by a computer. This step can be facilitated by the radiopacity of one or more elements of endovascular device 1, as discussed above. For example, this may be accomplished with a computer vision model trained to recognize radiopaque elements.

Once both the target area and endovascular device 1 are located, in a step 1208, a determination is made regarding whether a particular portion of the endovascular device is positioned at the target area. This can be accomplished using techniques similar to those discussed with respect to steps 1206 and 1208.

If that is the case, then in a step 1210 curvature of the particular portion of endovascular device 1 is controlled to navigate to the target area. This can be accomplished by altering the curvature of selectively bendable portion 110 as discussed above. The particular portion of endovascular device 1 can be or include distal end 2, and selectively bendable portion 110 can be located at distal end 2. In these embodiments, distal end 2 does not transition beyond the target area.

In some embodiments, endovascular device 1 is configured as a guidewire for guiding a suitable medical device, such as an aspiration catheter to the target area. In other embodiments, endovascular device 1 is itself configured as a catheter (e.g., as a guidecatheter), for example.

After actions such as a medical treatment are completed, a second medical image can be taken to ensure a successful treatment. In addition, or in the alternative, a signal received from a sensor 108 disposed in endovascular device 1 can be used to determine completion of the action. Sensor 108 can be any suitable sensor, and, for example, can function to sense the presence of a clot, the type of clot, or the type of stenosis present in the blood vessel. After the action is completed, the curvature of endovascular device can be controlled to reverse or modify the initial curvature control step. This allows for removal of endovascular device 1.

In embodiments of endovascular device 1 with multiple selectively bendable portion 110, the initial process described above regarding controlling curvature of the particular portion can be done for each selectively bendable portion 110 using suitable imagery.

These steps can also be performed on a suitable computing device possessing a memory and a processor. In particular the evaluation of the imagery and determination of the relative positions of endovascular device 1 and the target area can be performed by suitable algorithms stored on memory and run by a processor.

Exemplary embodiments of the invention are further provided below.

### Example 1

An endovascular device comprising a tube comprising a selectively bendable portion, wherein at least a portion of the tube is configured to be inserted into a blood vessel; a plug disposed at a distal end of the tube, wherein the plug has a rounded tip configured to be inserted into the blood vessel; a washer disposed between the plug and the tube, the washer fixed with respect to the tube; a control element extending through at least a portion of the tube, a distal end of the control element connected to the washer, wherein movement of the control element relative to the tube is configured to cause the selectively bendable portion of the tube to bend; and a spacer disposed within the tube and placed between the tube and the control element, the spacer comprising at least one wire formed in a coil and configured to reduce sliding friction between the tube and the control element, and wherein the coil has a pitch at least 1.2 times a diameter of the wire.

### Example 2

An endovascular device, comprising: a tube comprising a selectively bendable portion, wherein at least a portion of the tube is configured to be inserted into a blood vessel; a plurality of slots formed in the tube, each of the slots disposed partially around the circumference of the tube; a control element extending through at least a portion of the tube, the control element connected to a distal portion of the selectively bendable portion, wherein movement of the control element relative to the tube is configured to cause the selectively bendable portion of the tube to bend; and a spacer disposed within the tube and placed between the tube and the control element, the spacer comprising at least one wire formed in a coil and configured to reduce sliding friction between the tube and control element, and wherein the coil has a pitch at least 1.2 times a diameter of the wire.

### Example 3

The endovascular device of example 2, further comprising a plug disposed at a distal end of the tube, wherein the plug has a rounded tip configured to be inserted into a blood vessel.

Example 4

The endovascular device of any one of examples 2 or 3, further comprising a washer disposed in the tube, the washer fixed with respect to the tube, wherein at least one of the control element and the spacer are connected to the washer.

### Example 5

The endovascular device of any one of the preceding examples, wherein the washer is fixed rotationally with respect to an axis of the tube such that application of torque to the control element causes the selectively bendable portion of the tube to bend.

### Example 6

The endovascular device of any one of the preceding examples, wherein a distal end of the spacer is connected to the washer.

### Example 7

The endovascular device of any one of the preceding examples, wherein the control element is configured to slide with respect to at least a portion of the tube.

### Example 8

The endovascular device of any one of the preceding examples, wherein the control element extends through the tube from the washer at the distal end of the tube to a proximal end of the tube.

### Example 9

The endovascular device of any one of the preceding examples, wherein the proximal end of the control element is connected to an actuator control.

### Example 10

The endovascular device of any one of the preceding examples, wherein the diameter of the control element tapers from the proximal end of the tube to the distal end of the tube.

### Example 11

The endovascular device of any one of the preceding examples, wherein a portion of the control element closer to the distal end of the tube has a rectangular cross-section, and a portion of the control element closer to a proximal end of the tube has a circular cross-section.

### Example 12

The endovascular device of any one of the preceding examples, wherein the spacer surrounds at least part of the control element circumferentially.

### Example 13

The endovascular device of any one of the preceding examples, wherein the spacer is at least partially formed from a radiopaque material.

### Example 14

The endovascular device of example 1, wherein the tube comprises a plurality of slots, each of the slots disposed partially around the circumference of the tube.

### Example 15

The endovascular device of any one of the preceding examples, wherein a first slot of the plurality of slots faces in the first direction, and a second slot of the plurality of slots faces in a second direction different from the first direction, such that a first extent of the first slot is greater than a second extent of the second slot. According to an embodiment, the plurality of slots are formed on a portion of the tube comprising the selectively bendable portion

### Example 16

The endovascular device of any one of the preceding examples, wherein the extent difference between the first slot and the second slot biases the selectively bendable portion to bend in the first direction when the wire is moved.

### Example 17

The endovascular device of any one of the preceding examples, wherein the first slot of the plurality of slots has a larger area than the second slot of the plurality of slots.

### Example 18

The endovascular device of any one of the preceding examples, wherein the first slot of the plurality of slots has a different shape than the second slot of the plurality of slots.

### Example 19

The endovascular device of any one of the preceding examples, wherein the second direction is oriented to face an opposite radial direction than the first direction.

### Example 20

The endovascular device of any one of the preceding examples, further comprising a second plurality of slots formed on a portion of the tube separate from the selectively bendable portion, wherein the second plurality of slots are distributed symmetrically about the tube, and wherein the second plurality of slots enhances the flexibility of the tube in the portion.

### Example 21

The endovascular device of any one of the preceding examples, wherein a first slot of the plurality of slots is rotationally offset with respect to an adjacent second slot of the plurality of slots such that ends of the first slot are not aligned with ends of the second slot.

### Example 22

The endovascular device of any one of the preceding examples, wherein the plurality of slots comprises at least two slots dispersed along the tube, and wherein each of the slots is rotationally interleaved with an adjacent slot.

### Example 23

The endovascular device of any one of the preceding examples, wherein each slot of the plurality of slots is rotationally offset from an adjacent slot of the plurality of slots by a predetermined angle.

### Example 24

The endovascular device of any one of the preceding examples, wherein each slot of the plurality of slots are formed with an elongated side, and wherein the elongated side is aligned perpendicular to an axis of the tube.

### Example 25

The endovascular device of any one of the preceding examples, wherein each slot of the plurality of slots are formed with an elongated side, and wherein the elongated side is aligned at a non-perpendicular angle to an axis of the tube.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way. Moreover, the examples described above do not limit the present disclosure to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art.

The use of the modifiers “approximately” or “about” in this disclosure are intended to indicate that the relevant element is subject to variation by a tolerance range. Unless otherwise defined, the use of these modifiers with respect to a unit of measure means a tolerance of plus or minus ten percent of the unit of measure. The use of these modifiers with respect to a description such as a shape is intended to allow for variations of that shape due to tolerance issues as would be understood to occur in the art in general.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Various features of the invention which are, for clarity, described in the contexts of separate embodiments may also be provided in combination in a single embodiment. Conversely, various features of the invention which are, for brevity, described in the context of a single embodiment may also be provided separately or in any suitable sub-combination.

The breadth and scope of the present invention should not be limited by any of the above-described exemplary embodiments, but should be defined only in accordance with the following claims and their equivalents.

1. An endovascular device, comprising:
a tube comprising a selectively bendable portion, wherein at least a portion of the tube is configured to be inserted into a blood vessel;
a plug disposed at a distal end of the tube, wherein the plug has a rounded tip configured to be inserted into the blood vessel;
a washer disposed between the plug and the tube, the washer fixed with respect to the tube;
a control element extending through at least a portion of the tube, a distal end of the control element connected to the washer, wherein movement of the control element relative to the tube is configured to cause the selectively bendable portion of the tube to bend; and
a spacer disposed within the tube and placed between the tube and the control element, the spacer comprising at least one wire formed in a coil and configured to reduce sliding friction between the tube and the control element, and wherein the coil has a pitch at least 1.2 times a diameter of the wire.

2. The endovascular device of claim 1, wherein the washer is fixed rotationally with respect to an axis of the tube such that application of torque to the control element causes the selectively bendable portion of the tube to bend.

3. The endovascular device of claim 1, wherein a distal end of the spacer is connected to the washer.

4. The endovascular device of claim 1, wherein the control element is configured to slide with respect to at least a portion of the tube.

5. The endovascular device of claim 1, wherein the control element extends through the tube from the washer at the distal end of the tube to a proximal end of the tube.

6. The endovascular device of claim 5, wherein the proximal end of the control element is connected to an actuator control.

7. The endovascular device of claim 5, wherein a diameter of the control element tapers from the proximal end of the tube to the distal end of the tube.

8. The endovascular device of claim 1, wherein a portion of the control element closer to the distal end of the tube has a rectangular cross-section, and a portion of the control element closer to a proximal end of the tube has a circular cross-section.

9. The endovascular device of claim 1, wherein the tube comprises a plurality of slots, each of the slots disposed partially around the circumference of the tube.

10. The endovascular device of claim 9, wherein a first slot of the plurality of slots faces in the first direction, and a second slot of the plurality of slots faces in a second direction different from the first direction, such that a first extent of the first slot is greater than a second extent of the second slot.

11. The endovascular device of claim 9, further comprising a second plurality of slots formed on a portion of the tube separate from the selectively bendable portion, wherein the second plurality of slots are distributed symmetrically about the tube, and wherein the second plurality of slots enhances the flexibility of the tube in the portion.

12. The endovascular device of claim 9, wherein a first slot of the plurality of slots is rotationally offset with respect to an adjacent second slot of the plurality of slots such that ends of the first slot are not aligned with ends of the second slot.

13. The endovascular device of claim 9, wherein each slot of the plurality of slots are formed with an elongated side, and wherein the elongated side is aligned perpendicular to an axis of the tube.

14. The endovascular device of claim 9, wherein each slot of the plurality of slots are formed with an elongated side, and wherein the elongated side is aligned at a non-perpendicular angle to an axis of the tube.

15. An endovascular device, comprising:
a tube comprising a selectively bendable portion, wherein at least a portion of the tube is configured to be inserted into a blood vessel;
a plurality of slots formed in the tube, each of the slots disposed partially around the circumference of the tube;
a control element extending through at least a portion of the tube, the control element connected to a distal portion of the selectively bendable portion, wherein movement of the control element relative to the tube is configured to cause the selectively bendable portion of the tube to bend; and
a spacer disposed within the tube and placed between the tube and the control element, the spacer comprising at least one wire formed in a coil and configured to reduce sliding friction between the tube and the control element, and wherein the coil has a pitch at least 1.2 times a diameter of the wire.

16. The endovascular device of claim 15, wherein the spacer surrounds at least part of the control element circumferentially.

17. The endovascular device of claim 15, wherein the spacer is at least partially formed from a radiopaque material.

18. The endovascular device of claim 15, wherein a first slot of the plurality of slots faces in the first direction, and a second slot of the plurality of slots faces in a second direction different from the first direction, such that a first extent of the first slot is greater than a second extent of the second slot.

19. The endovascular device of claim 18, wherein the extent difference between the first slot and the second slot biases the selectively bendable portion to bend in the first direction when the wire is moved.

20. The endovascular device of claim 18, wherein the first slot of the plurality of slots has a larger area than the second slot of the plurality of slots.

21. The endovascular device of claim 18, wherein the first slot of the plurality of slots has a different shape than the second slot of the plurality of slots.

22. The endovascular device of claim 18, wherein the second direction is oriented to face an opposite radial direction than the first direction.

23. The endovascular device of claim 15, further comprising a second plurality of slots formed on a portion of the tube separate from the selectively bendable portion, wherein the second plurality of slots are distributed symmetrically about the tube, and wherein the second plurality of slots enhances the flexibility of the tube in the portion.

24. The endovascular device of claim 15, wherein a first slot of the plurality of slots is rotationally offset with respect to an adjacent second slot of the plurality of slots such that ends of the first slot are not aligned with ends of the second slot.

25. The endovascular device of claim 24, wherein the plurality of slots comprises at least two slots dispersed along the tube, and wherein each of the slots is rotationally interleaved with an adjacent slot.

26. The endovascular device of claim 15, wherein each slot of the plurality of slots is rotationally offset from an adjacent slot of the plurality of slots by a predetermined angle.

27. The endovascular device of claim 15, wherein each slot of the plurality of slots are formed with an elongated side, and wherein the elongated side is aligned perpendicular to an axis of the tube.

28. The endovascular device of claim 15, wherein each slot of the plurality of slots are formed with an elongated side, and wherein the elongated side is aligned at a non-perpendicular angle to an axis of the tube.

29. The endovascular device of claim 15, further comprising a plug disposed at a distal end of the tube, wherein the plug has a rounded tip configured to be inserted into a blood vessel.

30. The endovascular device of claim 15, further comprising a washer disposed in the tube, the washer fixed with respect to the tube, wherein at least one of the control element and the spacer are connected to the washer.
